(19) United States
(12) Patent Application Publication
(10) Pub. No.: US 2020/0271960 A1
(43) Pub. Date: Aug. 27, 2020

(54) MODULARIZED TEMPLE OF SPECTACLES AND SMART EYEWEAR

(71) Applicant: INNOCEAN WORLDWIDE, INC., Seoul (KR)

(72) Inventors: Hyun Jung BAEK, Seoul (KR); In Jeong CHOI, Yongin-si (KR); Sung Bum HU, Seoul (KR)

(21) Appl. No.: 16/099,827

(22) PCT Filed: Feb. 2, 2018
(86) PCT No.: PCT/KR2018/001459

§ 371 Date: Nov. 8, 2018

(30) Foreign Application Priority Data
Dec. 27, 2017 (KR) 10-2017-0181325

(51) Int. Cl. G02C 11/00; G02C 5/14; A61B 5/024; A61B 5/00; G08B 21/04

(52) U.S. Cl. CPC G02C 11/10; G02C 5/14; A61B 5/02438; G02C 2200/08; A61B 5/746; G08B 21/0453; A61B 5/6803

(57) ABSTRACT

According to an exemplary embodiment of the present disclosure, a modularized temple of spectacles includes: a function unit configured to perform a preset function; a sensor unit provided to correspond to a function of the function unit and receiving a stimulus from the outside; an output unit outputting a signal corresponding to the function set in the function unit and the stimulus received by the sensor unit; and a battery providing power to an eyewear, wherein the modularized temple of spectacles is configured to be attachable to or detachable from an eyewear body of the eyewear. As a result, a user may replace and use modularized temple of spectacles configured to perform different functions depending on a user purpose.

### TECHNICAL FIELD

The present disclosure relates to a modularized temple of spectacles capable of being attached to and detached from an eyewear body and having a preset function, and a smart eyewear.

### BACKGROUND ART

Conventionally, an eyewear such as glasses, sunglasses, and goggles has been used only for a single function, such as a vision correction function or an ultraviolet (UV) protection function. However, today, a smart eyewear compositely having various functions such as a route guidance function, a photographing function, and an earphone function, in addition to the function of the existing eyewear, has been gradually commercialized. Since a user may use required functions of the smart eyewear only by wearing the smart eyewear without using an additional component, the smart eyewear has many advantages in terms of usability and portability. Therefore, multi-functional smart eyewears having various and high-level functions have been developed and released.

However, the currently used smart eyewear is heavier and more inconvenient in terms of fit than a general eyewear since it implements a plurality of functions in a complex manner. Particularly, the multi-functional smart eyewear has fatal disadvantages that a frame can not but be manufactured at a large thickness due to modules, sensors, a liquid crystal, and a battery size, which may hurt a user's fashion and an aesthetic design, and it is difficult to efficiently use a battery for a long time in order to operate a plurality of sensors.

### DISCLOSURE

### Technical Problem

An aspect of the present disclosure provides a modularized temple of spectacles and a smart eyewear capable of performing various functions and being lightened in terms of an entire weight by allowing the modularized temple of spectacles having only a single function to be attachable to or detachable from an eyewear body and allowing the modularized temple of spectacles corresponding to a desired function to be used according to a necessity through replacement.

### Technical Solution

According to an exemplary embodiment of the present disclosure, a modularized temple of spectacles includes: a function unit configured to perform a preset function; a sensor unit provided to correspond to a function of the function unit and receiving a stimulus from the outside; an output unit outputting a signal corresponding to the function set in the function unit and the stimulus received by the sensor unit; a communication unit communicating with a user terminal or a server; and a battery providing power to an eyewear, wherein the modularized temple of spectacles is configured to be attachable to or detachable from an eyewear body of the eyewear.

According to another exemplary embodiment of the present disclosure, a smart eyewear includes: an eyewear body including a frame and a lens; and a modularized temple of spectacles configured to be attachable to or detachable from the eyewear body, wherein the modularized temple of spectacles includes: a function unit configured to perform a preset function; a sensor unit provided to correspond to a function of the function unit and receiving a stimulus from the outside; an output unit outputting a signal corresponding to the function set in the function unit and the stimulus received by the sensor unit; a communication unit communicating with a user terminal or a server; and a battery providing power to an eyewear. Different modularized temples of spectacles implement different functions, and the temples of spectacles may be replaced and used according to a necessity of a user, such that a smart eyewear capable of providing various smart functions and being conveniently worn in daily life without increasing an entire size may be provided.

### Advantageous Effects

According to the modularized temple of spectacles and the smart eyewear according to the present disclosure, the modularized temple of spectacles having only a single function may be allowed to be attachable to and detachable from the eyewear body to accomplish lightness, and the user may directly select the respective modularized temples of spectacles and couple the selected modularized temples to the smart eyewear to provide various functions without increasing an entire size or weight of the smart eyewear.

### DESCRIPTION OF DRAWINGS

FIG. 1 is a view illustrating lenses and temples of spectacles of a smart eyewear according to an exemplary embodiment of the present disclosure.

FIG. 2 is a block diagram illustrating a modularized temple of spectacles according to an exemplary embodiment of the present disclosure.

FIG. 3 is a view illustrating components of a temple of spectacles of a smart eyewear for preventing drowsiness according to an exemplary embodiment of the present disclosure.

FIG. 4 is a block diagram illustrating the temple of spectacles of the smart eyewear for preventing drowsiness according to an exemplary embodiment of the present disclosure.

FIG. 5 is a view illustrating components of a temple of spectacles of a smart eyewear for a hearing impaired person according to an exemplary embodiment of the present disclosure.

FIG. 6 is a block diagram illustrating the temple of spectacles of the smart eyewear for a hearing impaired person according to an exemplary embodiment of the present disclosure.

FIG. 7 is a view illustrating components of a temple of spectacles of a smart eyewear having a foreign language interpretation function according to an exemplary embodiment of the present disclosure.

FIG. 8 is a block diagram illustrating the temple of spectacles of the smart eyewear having a foreign language interpretation function according to an exemplary embodiment of the present disclosure.

FIG. 9 is a view illustrating a temple of spectacles including terminals for transmission of a current and data and charging according to an exemplary embodiment of the present disclosure.

FIG. 10A is a view illustrating an internal structure of a lens of the smart eyewear according to an exemplary embodiment of the present disclosure when the lens is in an off-state.

FIG. 10B is a view illustrating an internal structure of the lens of the smart eyewear according to an exemplary embodiment of the present disclosure when the lens is in an on-state.

### BEST MODE

Hereinafter, exemplary embodiments of the present disclosure will be described in detail with reference to the accompanying drawings. It is to be noted that in giving reference numerals to components of the accompanying drawings, the same components will be denoted by the same reference numerals even though they are illustrated in different drawings. Further, in describing exemplary embodiments of the present disclosure, well-known constructions or functions will not be described in detail in the case in which it is decided that they may unnecessarily obscure the understanding of exemplary embodiments of the present disclosure.

In addition, terms ‘first’, ‘second’, A, B, (a), (b), and the like, will be used in describing components of exemplary embodiments of the present disclosure. These terms are used only in order to distinguish any component from other components, and features, sequences, or the like, of corresponding components are not limited by these terms. When it is mentioned that any component is “connected” or “coupled” to another component, it is to be understood that any component is directly connected or coupled to another component or is connected or coupled to another component with the other component interposed therebetween.

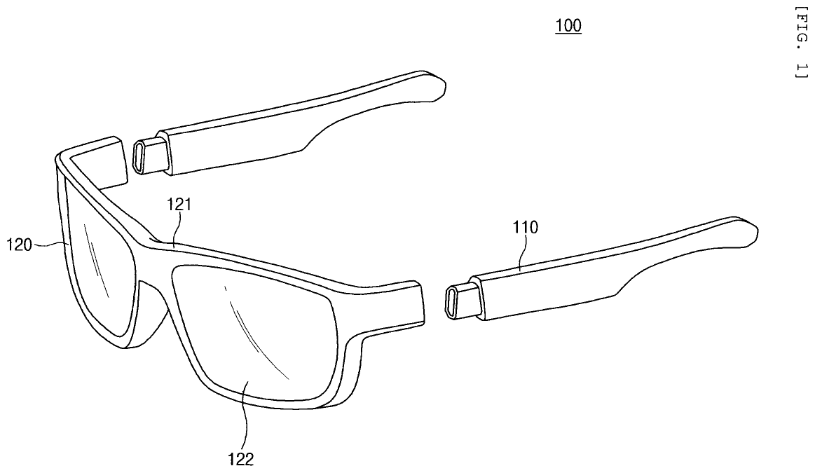

FIG. 1 is a view illustrating lenses and temples of spectacles of a smart eyewear according to an exemplary embodiment of the present disclosure.

Referring to FIG. 1, a smart eyewear 100 according to an exemplary embodiment of the present disclosure includes temples 110 of spectacles and an eyewear body 120. Here, the eyewear means all products worn on eyes and including lenses and temples of spectacles, such as glasses, goggles, sports glasses, protective goggles, sunglasses, and the like.

The modularized temples 110 of spectacles have a battery embedded thereto, and are modularized so as to be attachable to or detachable from the eyewear body 120. Particularly, the modularized temples 110 of spectacles have preset functions such as a drowsiness preventing function, a hearing impaired person assisting function, a foreign language interpretation function, and the like, mounted therein, and are configured to be coupled to the eyewear body 120 to implement the respective functions.

The eyewear body 120 includes a frame 121 and lenses 122. In addition, the eyewear body 120 may include an illumination sensor sensing illumination around a user as described below, and have a function of automatically adjusting transparency of the lenses depending on illumination sensed by the illumination sensor. That is, a signal for the illumination sensed by the illumination sensor is transmitted to the temples 110 of spectacles, and the transparency of the lenses 122 may be adjusted depending on a control signal received from the temples 110 of spectacles or a current or a voltage applied from the battery.

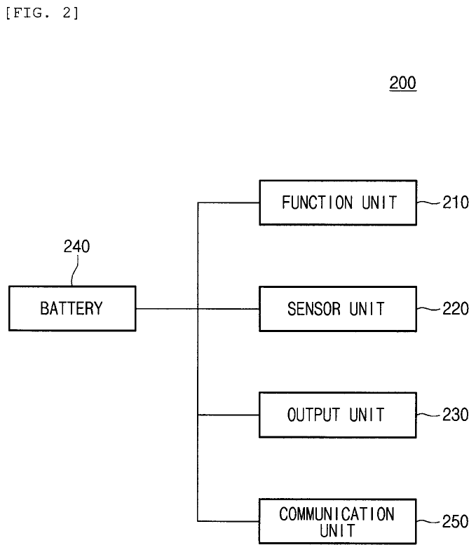

FIG. 2 is a block diagram illustrating a modularized temple 200 of spectacles according to an exemplary embodiment of the present disclosure.

Referring to FIG. 2, the modularized temple 200 of spectacles according to an exemplary embodiment of the present disclosure may include a function unit 210, a sensor unit 220, an output unit 230, a battery 240, and a communication unit 250.

The function unit 210 is configured to perform preset functions. For example, the function unit 210 serves to perform the respective functions mounted in the modularized temple 200 of spectacles, such as the drowsiness preventing function, the hearing impaired person assisting function, the foreign language interpretation function, and the like.

The sensor unit 220 senses signals corresponding to the preset functions of the function unit 210. For example, a temple of spectacles having the drowsiness preventing function may include a heart rate sensor, a motion sensor, a gyro sensor, and the like, so as to sense a heart rate, eye blink, a neck bending motion, and the like, of a user, and a temple of spectacles for a hearing impaired person may include a microphone so as to sense a danger sound around the user, or the like.

The output unit 230 outputs the signals corresponding to the preset functions of the function unit 210. For example, the output unit 230 of the temple 200 of spectacles having the drowsiness preventing function may be configured to output an alarm signal such as vibrations, a sound, or the like, when drowsiness is sensed, and the output unit 230 of the temple of spectacles for a hearing impaired person may be configured to output an alarm signal such as vibrations, light, or the like, when the danger sound around the user is sensed.

The battery 240 provides power to the eyewear. That is, the battery is configured to supply the power to the eyewear body as well as the function unit 210, the sensor unit 220, and the output unit 230 described above. That is, the battery supplies power required for the respective components of the modularized temple 200 of spectacles to perform functions, and applies a current or a voltage to the eyewear body to enable adjustment of the transparency of the lenses depending on the illumination.

The communication unit 250 communicates with a user terminal or a server. For example, the communication unit 250 may communicate with the user terminal such as a smartphone, a tablet, or the like, to perform detailed setting for each function such as sensitivity of the sensors, vibration strength, and the like, through an application of the smartphone. In addition, the communication unit 250 may communicate with the server (not illustrated) to be used to transmit and receive data on interpretation contents of spectacles for a travel or sound analysis data of spectacles for a hearing impaired person.

Meanwhile, the modularized temple 200 of spectacles according to an exemplary embodiment of the present disclosure may be designed so that the function unit 210, the sensor unit 220, and the output unit 230 are disposed in a left (or right) temple and the battery 240 and the communication unit 250 are disposed in a right (or left) temple. This is to easily manufacture the modularized temple 200 of spectacles, the function unit 210, the sensor unit 220, and the output unit 230 that need to be differently designed for each function may be included in the left (or right) temple of which a design is changed, and the battery 240 and the communication unit 250 common to all functions may be included in the right (or left) temple and be manufactured without a change in a design.

In addition, the modularized temple 200 of spectacles further includes a connector (not illustrated) for connecting the modularized temple of spectacles to the eyewear body, and may be configured to be physically connected to the eyewear body through the connector to allow an electrical signal and a current to be transmitted.

The exemplary embodiment described above may be implemented in any of a plurality of manners. For example, exemplary embodiments made by designing the technology disclosed herein may be implemented by hardware, software, or a combination thereof. When the exemplary embodiment is implemented by the software, software codes may be executed by any appropriate processor or a set of processors regardless of whether they are provided to a single computer or are distributed among a plurality of computers. That is, the function unit 210, and the like, may allow the processor to execute a control program stored in a memory to perform the function. The same goes for the following exemplary embodiments.

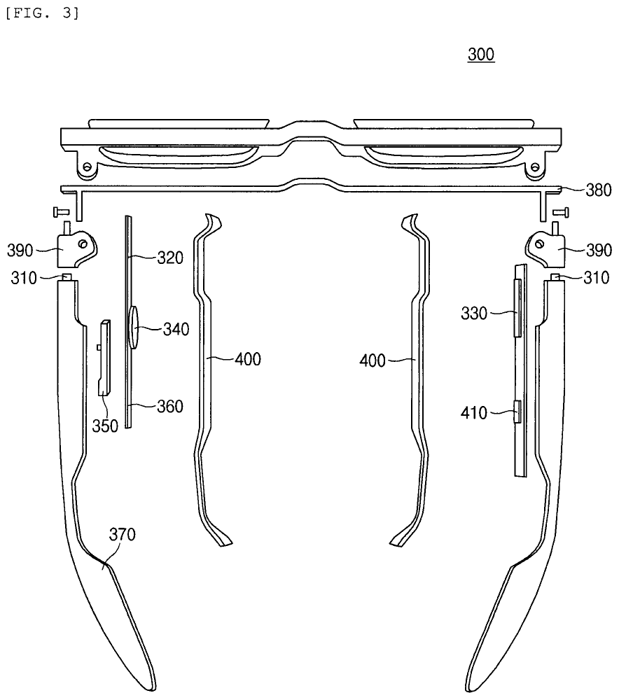

Hereinafter, examples of components of modularized temples of spectacles of a smart eyewear for each function according to an exemplary embodiment of the present disclosure will be described with reference to FIGS. 3 to 8. However, functions to be described below are only examples of functions of modularized temples of spectacles according to an exemplary embodiment of the present disclosure, and components of the present disclosure are not limited to the following examples.

FIG. 3 is a view illustrating components of a smart eyewear for preventing drowsiness according to an exemplary embodiment of the present disclosure. A temple 300 of spectacles for preventing drowsiness senses whether or not a user of the smart eyewear blinks his/her eyes depending on drowsiness through a motion sensor 320 provided as the sensor unit 220, measures a heart rate using a heart rate sensor 340 provided as the sensor unit 220 to sense a change depending on a decrease in the heart rate, or decides whether or not the user of the smart eyewear drowses by sensing a neck bending motion of the user through a gyro sensor 360 provided as the sensor unit 220, and generates a warning alarm as vibrations, a sound, or the like, through an alarm device provided as the output unit 230 to prevent a danger of the user of the smart eyewear depending on the drowsiness.

Referring to FIG. 3, the temple 300 of spectacles of a smart eyewear for preventing drowsiness includes a micro universal serial bus (USB) port 310, a battery 330, the motion sensor 320, the heart rate sensor 340, the gyro sensor 360, a main central processing unit (CPU) 350, a vibration device 370, and a communication device 410. The temple 300 of spectacles of a smart eyewear may be physically and electrically coupled to or decoupled from an eyewear body through a terminal, as illustrated in FIG. 3.

The micro USB port 310 corresponds to a connector for coupling the temple 300 of spectacles for preventing drowsiness to the eyewear body. That is, the temple 300 of spectacles of a smart eyewear is physically coupled to the eyewear body through the micro USB port 310, and may perform electrical connection for the purpose of supply of power of the battery 330 and transmission and reception of electrical signals (a sensed signal from the eyewear body, a control signal of the temple of spectacles, and the like). A mounted portion 390 in which the USB port 310 is to be mounted is formed in the eyewear body, and physical coupling may be performed through the mounted portion. However, the present disclosure is not limited thereto, and a member complementing the physical coupling may be further formed.

The battery 330 provides power required for operating the respective components such as the sensors 320, 340, and 360, the main CPU 350, and the like, in the temple 300 of spectacles for preventing drowsiness. In addition, as described above, the battery 330 may serve to transmit the current to the eyewear body to generate an electrical stimulus required for automatically adjusting the transparency of the lenses.

The motion sensor 320 is included as an example of the sensor unit 220 in FIG. 2. The motion sensor 320 senses whether or not the user of the smart eyewear blinks his/her eyes depending on the drowsiness. In detail, the motion sensor may decide that the user is in a drowsy state when it senses that eyes of the user of the smart eyewear are closed for a predetermined time or more. The heart rate sensor 340 is included as an example of the sensor unit 220 in FIG. 2. The heart rate sensor 340 measures a change in the heart rate of the user of the smart eyewear depending on the drowsiness. In detail, the heart rate sensor may serve to prevent the drowsiness by sensing a decrease in the heart rate generated immediately before the user of the smart eyewear drowses. Therefore, for example, an accident caused by the drowsiness during driving may be prevented.

The gyro sensor 360 is included as an example of the sensor unit 220 in FIG. 2. The gyro sensor 360 senses the neck bending motion (posture) of the user of the smart eyewear due to the drowsiness. That is, the gyro sensor may decide that the user is in a drowsy state when it senses that the user of the smart eyewear bends his/her neck for a predetermined time or more.

The main CPU 350 receives the power from the battery 330, and serves to generally control the sensors 320, 340, and 360, the vibration device 370, and the like, of the temple 300 of spectacles. In addition, the main CPU may also serve to transmit the control signal, or the voltage or the current for adjusting the transparency of the lenses to the eyewear body. That is, the main CPU 350 may serve as the function unit 210 in FIG. 2.

The vibration device 370 is an example of an alarm device which is the output unit 230 in FIG. 2. That is, when the drowsiness of the user of the smart eyewear is sensed through the sensors described above, that is, the motion sensor 320, the heart rate sensor 340, and the gyro sensor 360, the vibration device 370 serves to generate a warning alarm by vibrations to wake up the user of the smart eyewear. In this case, the temple 300 of spectacles of a smart eyewear for preventing drowsiness may include a light emitting diode (LED) device, a speaker device, or the like, instead of the vibration device 370 to generate a warning alarm for the user through light, a sound signal, or the like.

Meanwhile, in FIG. 3, an auxiliary frame 380 is configured in the same form as that of a frame of the eyewear body, is physically connected to the frame of the eyewear body and the temple 300 of spectacles, and includes a circuit for transferring an electrical signal. That is, the auxiliary frame 380 serves as a path transferring the electrical signal between the modularized temple 300 of spectacles and the eyewear body. For example, the control signal may be transmitted from the main CPU 350 to the lenses of the eyewear body through the auxiliary frame 380. A circuit configuration in the auxiliary frame 380 described above may also be configured to be included in the frame of the eyewear body.

The mounted portion 390 of the eyewear body is a portion through which the temple 300 of spectacles is physically and electrically connected to the eyewear body. That is, the USB port 310 of the modularized temple 300 of spectacles is coupled to the mounted portion 390 of the eyewear body, such that the control signal, the voltage, or the current may be applied from the temple of spectacles to the eyewear body or signals may be transmitted and received between two temples 300 of spectacles through the auxiliary frame 380.

Covers 400 of the temple of spectacles serve to protect internal components such as the battery, the sensors, or the like, of the two temples 300 of spectacles. In addition, the covers 400 may include circuits as in the auxiliary frame 380 described above to serve to transmit signals from the sensors 320, 340, and 360 or the main CPU 350 included in the temple of spectacles to the eyewear body or an opposite temple of spectacles.

The communication device 410 serves to communicate with a user terminal so as to allow the user to set sensitivity of the sensors 320, 340, and 360 or vibration strength of the vibration device 370 using a smart application, or the like, of the user terminal such as a smartphone, a tablet, or the like.

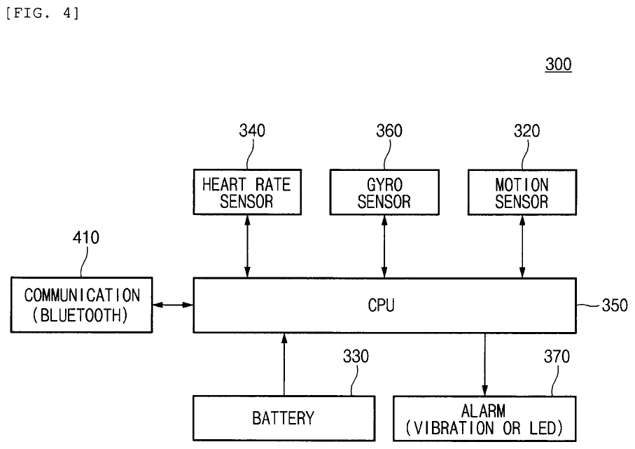

FIG. 4 is a block diagram illustrating the temple 300 of spectacles of the smart eyewear for preventing drowsiness according to an exemplary embodiment of the present disclosure. The same components as those of FIG. 3 will be denoted by the same reference numerals.

Referring to FIG. 4, when the motion sensor 320, the heart rate sensor 340, and the gyro sensor 360 sense the eye blink, the decrease in the heart rate, and the neck bending motion of the user of the smart eyewear due to the drowsiness as described above, the motion sensor 320, the heart rate sensor 340, and the gyro sensor 360 transmit sensed signals to the main CPU 350. When the main CPU 350 receives the sensed signals from the sensors 320, 340, and 360, the main CPU 350 may transmit a control signal to the vibration device (or the LED device, the speaker device, or the like) to provide the warning alarm through vibrations (or a screen, a sound, or the like), thereby preventing the drowsiness of the user of the smart eyewear. In this case, as described above, the user may communicate with the communication device 410 using the smartphone, or the like, to appropriately set the sensitivity of the sensors, the vibration strength, or the like to desired strength.

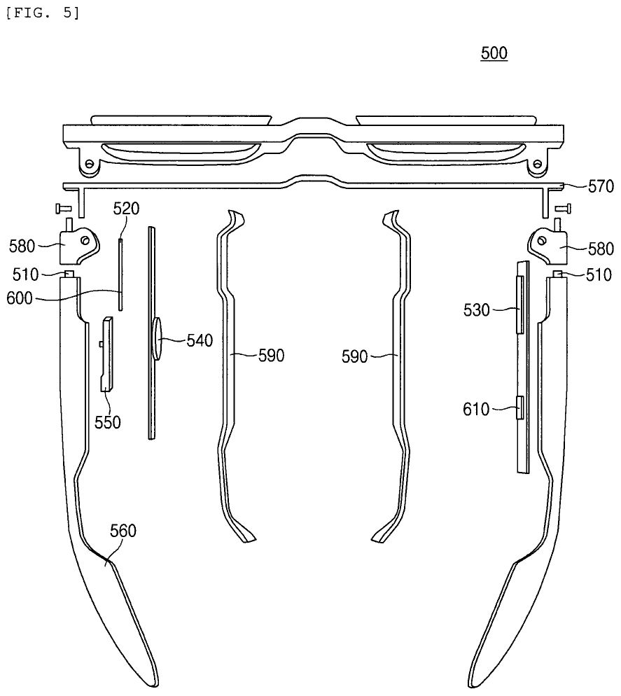

FIG. 5 is a view illustrating components of a smart eyewear for a hearing impaired person according to an exemplary embodiment of the present disclosure. A temple 500 spectacles for a hearing impaired person senses a sound around the user through a microphone 540 provided as the sensor unit 220, and decides that the sensed sound is a danger sound to generate a warning alarm such as vibrations, light, or the like, through an alarm device provided as the output unit 230, thereby transferring information on a danger to the user of the smart eyewear.

Referring to FIG. 5, the temple 500 of spectacles of a smart eyewear for a hearing impaired person includes a micro USB port 510, a battery 530, the microphone 540, an operational amplifier 600, an analog to digital converter (ADC) 520, a main CPU 550, a vibration device 560, and a communication device 610. An overlapping description for the same components as those of FIG. 3 will be omitted. That is, the micro USB port 510, the battery 530, an auxiliary frame 570, a mounted portion 580 of an eyewear body, and covers 590 of the temple of spectacles are components having the same functions as those described in FIG. 3.

The microphone 540 is a device mounted in the modularized temple 500 of spectacles and sensing a sound around the user of the smart eyewear through a micro electro mechanical system (MEMS) technology.

The operational amplifier 600 serves to amplify a sensed sound signal when the sound around the user of the smart eyewear is sensed through the microphone 540.

The ADC 520 is an analog-to-digital converter converting an analog signal into a digital signal. That is, the ADC converts an analog sound signal around the user, sensed by the microphone 540 into a digital signal, transmits the digital signal to the main CPU 550, and allows a control signal to be output depending on a received signal.

The main CPU 550 receives power from the battery 530, and serves to analyze the sound signal and control the vibration device 560, and the like. That is, the main CPU distinguishes and recognizes a specific danger sound such as a horn sound, a crash sound, a siren sound, or the like, of a vehicle through a frequency analysis of the sound signal, and controls the vibrations device 560.

The vibration device 560 provides a warning alarm by vibrations to the user of the smart eyewear when it is decided that the sensed sound around the user is the horn sound or the danger sound. That is, when the danger sound around the user is sensed, the vibration device serves as an alarm device receiving the control signal by the main CPU 550 and generating an alarm for the user. In this case, the temple 500 of spectacles of a smart eyewear for a hearing impaired person may also include an LED device, or the like, instead of the vibration device 560 to generate a warning alarm for the user of the smart eyewear through a light signal, or the like.

The communication device 610 serves to transmit and receive analysis data on various sound signals sensed through the microphone 540 to and from the server. That is, the communication device 610 receives the analysis data on the various sound signals from the server, and may decide whether or not the sensed sound around the user is any danger signal and accurately distinguish and decide the various sound signals.

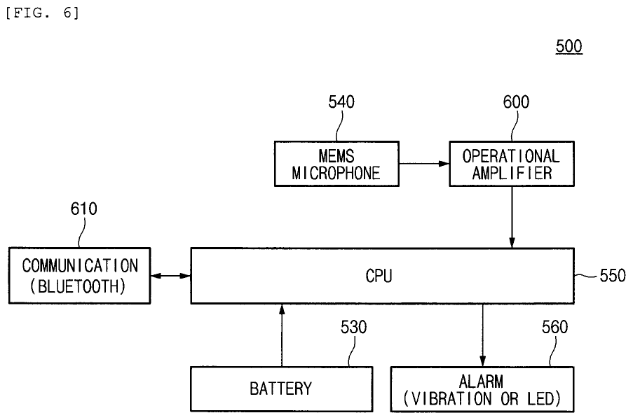

FIG. 6 is a block diagram illustrating the temple 500 of spectacles of the smart eyewear for a hearing impaired person according to an exemplary embodiment of the present disclosure. The same components as those of FIG. 5 will be denoted by the same reference numerals.

When the sound around the user of the smart eyewear is sensed through the MEMS microphone 540, the sensed sound signal is amplified by the operational amplifier 600. In addition, the CPU 550 performs the frequency analysis on the sensed sound signal by a fast Fourier transform (FFT) method, and distinguishes the horn sound and the danger sound depending on frequency characteristics of the sound.

In this case, the communication device 610 receives sound analysis data from an external server, and allows accurate information on the sound to be added. Therefore, in the case in which it is decided that the sensed sound is the horn sound or the danger sound, as described above, the communication device transmits the control signal to the vibration device 560 (or the LED device, or the like) to provide the warning alarm through the vibrations (or the screen, or the like), thereby making it possible to transfer information on danger elements such as a vehicle around the user of the smart eyewear, and the like, to the user of the smart eyewear, particularly, the hearing impaired person. Therefore, it is possible to prevent a danger of an accident.

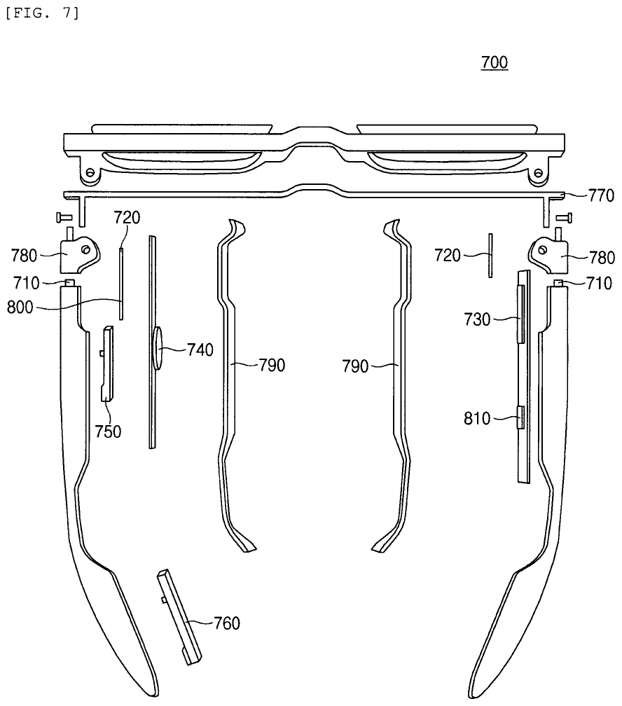

FIG. 7 is a view illustrating components of a smart eyewear having a foreign language interpretation function according to an exemplary embodiment of the present disclosure. A temple 700 of spectacles having a foreign language interpretation function includes a microphone 740 as a sensor unit and a speaker as an output unit, further includes a communication unit (not illustrated) communicable with the server, recognizes a language around the user by the microphone 740 and transfers the interpreted language generated by interpreting the language using interpretation data recorded in the server to the user through the speaker 760.

Referring to FIG. 7, the temple 700 of spectacles of a smart eyewear having a foreign language interpretation function includes a micro USB port 710, a battery 730, the microphone 740, an operational amplifier 800, an ADC 720, a main CPU 750, a speaker system 760, and a communication device 810. Likewise, an overlapping description for the same components as those of FIGS. 3 and 5 will be omitted. The micro USB port 710, the battery 730, the microphone 740, the operational amplifier 800, the ADC 720, the main CPU 750, an auxiliary frame 770, a mounted portion 780 of an eyewear body, covers 790 of the temple of spectacles, and the communication device 810 are components having the same functions as those described in FIGS. 3 and 5.

The speaker system 760 is a device interpreting the language recognized through the MEMS microphone 740 and transferring the interpreted language to the user of the smart eyewear. That is, when the MEMS microphone 740 recognizes the language in the sound around the user of the smart eyewear, the speaker system serves to transfer the interpreted language generated by the server interpreting the recognized language to the user through a communication module.

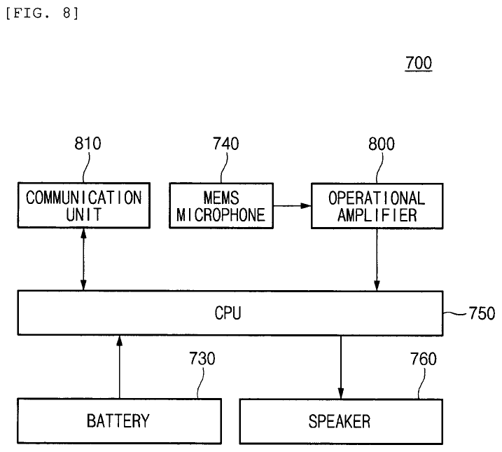

FIG. 8 is a block diagram illustrating the temple 700 of spectacles of the smart eyewear having a foreign language interpretation function according to an exemplary embodiment of the present disclosure. The same components as those of FIG. 7 will be denoted by the same reference numerals.

When the language around the user of the smart eyewear is recognized through the MEMS microphone 740, the sensed sound signal is amplified by the operational amplifier 800. The recognized local language is transmitted to the server through the communication device 810. In this case, the server interprets the recognized language using the interpretation data recorded in the server, and the interpreted language is again transmitted to the CPU 750. The CPU 750 receives the interpreted language data, converts the received language data into audio data, and transmits the audio data to the speaker 760, and the speaker 760 provides the interpreted language as an audio to the user of the smart eyewear.

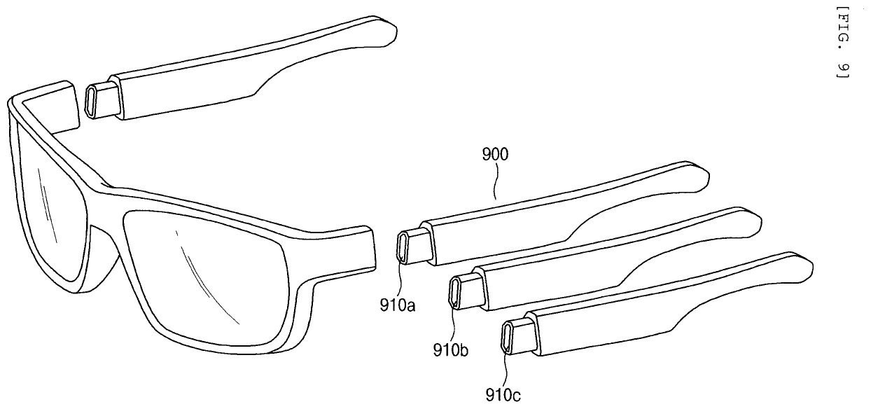

FIG. 9 is a view illustrating a temple of spectacles including terminals for transmission of a current and data and charging according to an exemplary embodiment of the present disclosure. A plurality of modularized temples 900 of spectacles illustrated in FIG. 9 include terminals 910a to 910c that physically couple the plurality of modularized temples 900 of spectacles to an eyewear body, may transmit electrical signals, and are inserted into a charger to charge batteries. In addition, the plurality of modularized temples of spectacles illustrated in FIG. 9 may be modularized temples of spectacles having different functions, and may be replaced anytime depending on functions desired by a user and be attached to the eyewear body.

Referring to FIG. 9, the terminals 910a to 910c for transmission of a current and a control signal and charging may be directly inserted into a charger of a general Android smartphone including a popularly used charging terminal, for example, Micro 5Pin to charge the batteries. In addition, the current and the control signal may be transmitted to the eyewear body through the terminals to change an electrical stimulus depending on the illumination around the user, thereby adjusting the transparency of the lenses.

In addition, the modularized temples of spectacles may be configured to include at least one of an LED module, a speaker module, a camera module, a global positioning system (GPS) module, a hearing aid module, a pulse detector module, a pupil monitor module, a wireless communication module, a display module, and other sensor modules. The modularized temples of spectacles further include the modules described above, in addition to the basic components of the modularized temple of spectacles described in FIG. 2, such that the smart eyewear according to an exemplary embodiment of the present disclosure may provide various functions such as a function performing audio based automatic memorization depending on a mobile phone use danger during driving, a function performing notification depending on acceleration or deceleration driving and recoding a driving habit, a function having a camera enabling photographing and image capturing, a function measuring and analyzing conditions (a heart rate, an oxygen saturation, and the like) during exercise or life in a state in which the various functions are included in the modularized temple of spectacles.

Particularly, the modularized temples of spectacles having the various functions as described above are manufactured in different colors depending on the functions, such that the user of the smart eyewear may easily distinguish and find the temples of spectacles having functions desired by him/her.

As described above, according to the modularized temple of spectacles and the smart eyewear according to the present disclosure, the modularized temple of spectacles having only a single function is allowed to be attachable to and detachable from the eyewear body, such that the various functions may be performed, and an entire weight may not be increased, thereby accomplishing lightness.

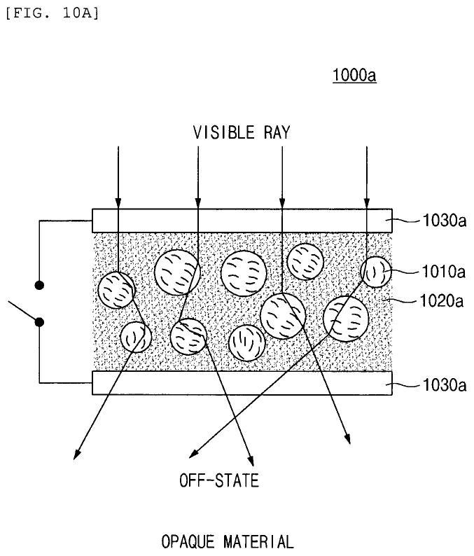

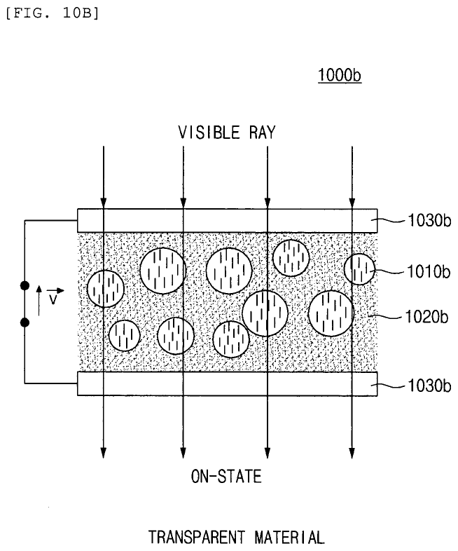

Hereinafter, a method in which the smart eyewear according to an exemplary embodiment of the present disclosure automatically adjusts transparency will be described with reference to FIGS. 10A and 10B.

FIGS. 10A and 108 are views illustrating a principle in which transparency of lenses included in the eyewear body of the smart eyewear according to an exemplary embodiment of the present disclosure is adjusted depending on illumination. The frame of the eyewear body of the smart eyewear according to the present disclosure may include the illumination sensor sensing the illumination around the user, and the lens may automatically adjust the transparency depending on the illumination sensed by the illumination sensor included in the frame. In addition, the lens of the smart eyewear according to an exemplary embodiment of the present disclosure may be formed of a polymer dispersed liquid crystal display element. A polymer dispersed liquid crystal is an element in which a plurality of liquid crystal dispersed particles having a micro size are dispersed in a polymer matrix to be adjustable in a transparent or opaque state by an electric field without using a polarizer.

FIG. 10A is a view illustrating an internal structure of a lens of the smart eyewear according to an exemplary embodiment of the present disclosure when the lens is in an off-state. The lens 1000a is in a state in which it is basically surrounded by conductive indium tin oxide (ITO) films 1030a and liquid crystal dispersed particles 1010a are arranged in any directions in a polymer matrix 1020a between the conductive ITO films 1030a, as illustrated in FIG. 10A. Particularly, in a state in which a voltage is not applied to the lens, the liquid crystal dispersed particles 1010a existing in the polymer matrix 1020a are irregularly arranged, such that a difference is generated between an effective refractive index of the liquid crystal dispersed particles 1010a and an effective refractive index of the polymer matrix 1020a. As a result, incident visible rays are scattered, such that the lens 1000a in the off-state shows opaque characteristics.

Meanwhile, FIG. 10B is a view illustrating an internal structure of the lens of the smart eyewear according to an exemplary embodiment of the present disclosure when the lens is in an on-state. When the lens 1000b included in the eyewear body becomes the on-state by a voltage applied to the lens 1000b through the battery of the temple of spectacles described above, liquid crystal dispersed particles 1010b in a polymer matrix 1020b are aligned in one direction, such that an effective refractive index of the liquid crystal dispersed particles 1010b and an effective refractive index of the polymer matrix 1020b become equal to each other. As a result, incident visible rays are not scattered, but are transmitted, such that the lens 1000b in the on-state shows transparent characteristics.

As described above, the transparency of the lens may be adjusted by adjusting the voltage applied to the lens of the smart eyewear. That is, the transparency of the lens of the smart eyewear may be optimized in real time by adjusting an electrical stimulus supplied from the battery depending on the illumination around the user, sensed by the illumination sensor. In addition, in the case in which the user does not want an automatic color change, the user may change a mode into a manual mode and then manually adjust the transparency of the lens. In this case, the modularized temple of spectacles may further include an input unit such as a switch, or the like, that may manually adjust the transparency of the lens.

In addition, although a case in which the polymer dispersed liquid crystal is used in the lens is described in the present exemplary embodiment, the present disclosure is not limited thereto. That is, another component may also be used as long as the transparency of the lens may be adjusted by applying a voltage or a current from the outside to the lens. Meanwhile, the polymer dispersed liquid crystal itself may be used as the lens or the polymer dispersed liquid crystal may be used in a structure in which it is formed on a front surface or a rear surface of the lens.

Although it has been described that all components configuring the exemplary embodiment of the present disclosure are combined with each other as one component or are combined and operated with each other as one component, the present disclosure is not necessarily limited to the abovementioned exemplary embodiment. That is, all the components may also be selectively combined and operated with each other as one or more components without departing from the scope of the present disclosure. In addition, although each of all the components may be implemented by one independent hardware, some or all of the respective components may be selectively combined with each other to be implemented by a computer program. Codes and code segments configuring the computer program may be easily inferred by those skilled in the art to which the present disclosure pertains. The computer program is stored in computer readable media and is read and executed by a computer, thereby making it possible to implement the exemplary embodiment of the present disclosure. An example of the computer readable media may include magnetic recording media, optical recording media, carrier wave media, and the like.

In addition, hereinabove, the terms “include”, “configure”, “have”, or the like, are to be interpreted to imply the inclusion of other components rather than the exclusion of other components, since they mean that a corresponding component may be included unless particularly described otherwise. Unless defined otherwise, all the terms including technical and scientific terms have the same meaning as meanings generally understood by those skilled in the art to which the present disclosure pertains. Generally used terms such as terms defined in a dictionary should be interpreted as the same meanings as meanings within a context of the related art and should not be interpreted as ideally or excessively formal meanings unless clearly defined in the present disclosure.

The spirit of the present disclosure has been illustratively described hereinabove. It will be appreciated by those skilled in the art that various modifications and alterations may be made without departing from the essential characteristics of the present disclosure. Accordingly, exemplary embodiments disclosed in the present disclosure are not to limit the spirit of the present disclosure, but are to describe the spirit of the present disclosure. The scope of the present disclosure is not limited to these exemplary embodiments. The scope of the present disclosure should be interpreted by the following claims, and it should be interpreted that all the spirits equivalent to the following claims fall within the scope of the present disclosure.

1. A modularized temple of spectacles, comprising:
a function unit configured to perform a preset function;
a sensor unit provided to correspond to a function of the function unit and receiving a stimulus from the outside;
an output unit outputting a signal corresponding to the function set in the function unit and the stimulus received by the sensor unit;
a communication unit communicating with a user terminal or a server; and
a battery providing power to an eyewear, wherein the modularized temple of spectacles is configured to be attachable to or detachable from an eyewear body of the eyewear, and provides the preset function of the modularized temple of spectacles to a user of the eyewear.

2. The modularized temple of spectacles according to claim 1, further comprising a connector for connecting the modularized temple of spectacles to the eyewear body,
wherein the modularized temple of spectacles is configured to be physically connected to the eyewear body through the connector to allow an electrical signal to be transmitted.

3. The modularized temple of spectacles according to claim 1, wherein a heart rate sensor is provided as the sensor unit, and an alarm device is provided as the output unit, and
the modularized temple of spectacles measures a heart rate of the user by the heart rate sensor, and generates a warning alarm for the user through the alarm device when a change depending on a decrease in the heart rate is sensed.

4. The modularized temple of spectacles according to claim 1, wherein a gyro sensor is provided an the sensor unit, and an alarm device is provided as the output unit, and
the modularized temple of spectacles senses a neck bending motion of the user by the gyro sensor, and generates a warning alarm for the user through the alarm device.

5. The modularized temple of spectacles according to claim 1, wherein a motion sensor is provided as the sensor unit, and an alarm device is provided as the output unit, and
the modularized temple of spectacles senses whether or not the user blinks his/her eyes by the motion sensor, and generates a warning alarm for the user through the alarm device.

6. The modularized temple of spectacles according to claim 1, wherein a microphone is provided as the sensor unit, and an alarm device is provided as the output unit, and
the modularized temple of spectacles senses a horn sound or a danger sound by the microphone, converts a sensed sound signal into a vibration signal or a light signal, and generates a warning alarm for the user through the alarm device.

7. The modularized temple of spectacles according to claim 1, wherein a microphone is provided as the sensor unit, and a speaker is provided as the output unit, and
the modularized temple of spectacles recognizes a language around the user by the microphone, and transfers an interpreted language generated by interpreting the language using interpretation data recorded in the server to the user through the speaker.

8. The modularized temple of spectacles according to claim 1, wherein an illumination sensor is provided as the sensor unit, and the output unit is configured to output an electrical signal depending on illumination, and
the modularized temple of spectacles adjusts transparency of a lens of the eyewear by controlling the electrical signal from the output unit depending on illumination around the user when the illumination around the user is sensed by the illumination sensor.

9. The modularized temple of spectacles according to claim 2, wherein the battery of the modularized temple of spectacles is chargeable through the connector.

10. A smart eyewear comprising:
an eyewear body including a frame and a lens; and
a modularized temple of spectacles configured to be attachable to or detachable from the eyewear body,
wherein the modularized temple of spectacles includes:
a function unit configured to perform a preset function;
a sensor unit provided to correspond to a function of the function unit and receiving a stimulus from the outside;
an output unit outputting a signal corresponding to the function set in the function unit and the stimulus received by the sensor unit;
a communication unit communicating with a user terminal or a server; and
a battery providing power to an eyewear.

11. The smart eyewear according to claim 10, wherein the modularized temple of spectacles further includes a connector for connecting the modularized temple of spectacles to the eyewear body, and
the modularized temple of spectacles is configured to be physically connected to the eyewear body through the connector to allow an electrical signal to be transmitted.

12. The smart eyewear according to claim 10, wherein a heart rate sensor is provided as the sensor unit, and an alarm device is provided as the output unit, and
the modularized temple of spectacles measures a heart rate of a user by the heart rate sensor, and generates a warning alarm for the user through the alarm device when a change depending on a decrease in the heart rate is sensed.

13. The smart eyewear according to claim 10, wherein a gyro sensor is provided an the sensor unit, and an alarm device is provided as the output unit, and
the modularized temple of spectacles senses a neck bending motion of a user by the gyro sensor, and generates a warning alarm for the user through the alarm device.

14. The smart eyewear according to claim 10, wherein a motion sensor is provided as the sensor unit, and an alarm device is provided as the output unit, and
the modularized temple of spectacles senses whether or not a user blinks his/her eyes by the motion sensor, and generates a warning alarm for the user through the alarm device.

15. The smart eyewear according to claim 10, wherein a microphone is provided as the sensor unit, and an alarm device is provided as the output unit, and
the modularized temple of spectacles senses a horn sound or a danger sound by the microphone, converts a sensed sound signal into a vibration signal or a light signal, and generates a warning alarm for a user through the alarm device.

16. The smart eyewear according to claim 10, wherein a microphone is provided as the sensor unit, and a speaker is provided as the output unit, and
the modularized temple of spectacles recognizes a language around a user by the microphone, and transfers an interpreted language generated by interpreting the language using interpretation data recorded in the server to the user through the speaker.

17. The smart eyewear according to claim 11, wherein the battery of the modularized temple of spectacles is chargeable through the connector.
